(19) United States
(12) Patent Application Publication
(10) Pub. No.: US 2024/0197154 A1
(43) Pub. Date: Jun. 20, 2024

(54) DEPLOYABLE FLEXIBLE OVERTUBE

(71) Applicant: Ergami Endoscopy, Brooklyn, NY (US)

(72) Inventor: Hrishikesh Vishvas DEO, Brooklyn, NY (US)

(21) Appl. No.: 18/541,689

(22) Filed: Dec. 15, 2023

Related U.S. Application Data
(60) Provisional application No. 63/433,360, filed on Dec. 16, 2022.

(51) Int. Cl. A61B 1/00; A61B 1/01

(52) U.S. Cl. CPC A61B 1/00154; A61B 1/01

(57) ABSTRACT

A flexible overtube and method of deploying the overtube is presented such that the overtube is configured to guide an endoscope to an in vivo deployment location in a patient's colon. A fixation balloon may be provided at the distal end of the overtube to fix the overtube in place. The packaging of a flexible overtube and fixation balloon may take the form of an origami fold patterns or patterns derived from stable inextensional post-buckling patterns of thin-walled cylinders under axial compression or combined axial-torsional loading. Such folding will result in the overtube and balloon being efficiently packaged in a fold containment mechanism. The unfolding progressive deployment of the flexible overtube may proceed from the proximal end to the distal end of the overtube. This packaging offers a convenient way to deploy the flexible overtube and deliver the propulsion and steering mechanism into the patient's colon.

### CROSS-REFERENCE TO RELATED APPLICATIONS

This application claims the benefit of U.S. Provisional Application No. 63/433,360, filed Dec. 16, 2022, the disclosure of which is hereby incorporated by reference herein in its entirety.

### TECHNICAL FIELD

The present disclosure generally relates to an apparatus for use in a colonoscopy procedure and, more particularly, to an initial deployment and fixation of a flexible overtube in the patient's colon. Once the overtube is deployed and fixated in the patient's colon, it receives and guides a colonoscope into the patient's colon with minimum effort.

### BACKGROUND

Colonoscopy is the most effective procedure for examination of the rectum and the colon. Close to 20 million colonoscopies are performed annually in the US alone. However, there are several challenges associated with colonoscopy such as: patient pain and discomfort; pain and endoscopy-related injuries in endoscopists and nurses; and prolonged and variable colonoscope insertion time.

Colonoscopy is a challenging procedure due to looping, which occurs when the flexible insertion tube of the colonoscope is advanced into the flexible and tortuous colon without corresponding progression of the distal tip, as shown in FIGS. 1A and 1B. Looping during colonoscope insertion leads to stretching of the colonic mucosa and causes patient pain and the need for sedation, which adds to patient risk, recovery time and procedure cost. Looping is also responsible for prolonged and variable insertion time, which has an adverse impact on scheduling and productivity. Colonoscopy also carries a small risk of colon perforation, which increases due to looping.

Colonoscopy requires considerable endoscopist training to learn and master. To prevent looping, endoscopists are required to perform repeated pushing, pulling, torquing and steering of the colonoscope, in awkward postures. Due to repetitive use of muscles, joints, and tendons; and contorting into awkward positions to traverse difficult turns in the colon, gastrointestinal (GI) endoscopists performing colonoscopy are at high risk of endoscopy-related injuries (ERIs). Several survey-based studies indicate that ERIs occur in up to 37-89% of practicing endoscopists.

Nurses are often required to apply abdominal pressure to prevent looping of the colonoscope, and to move sedated patients for gravity assistance during colonoscope insertion. The looping problem is more severe in obese patients due to redundant colon, and it is more challenging to move obese sedated patients. These factors place nurses at increased risk of repetitive stress injuries. Some studies indicate a prevalence of ERI in 85% of endoscopy nurses.

It is desirable to provide an improved technique for making colonoscope insertion less painful for the patients, endoscopists, and nurses. It may also be desirable to make the colonoscope insertion less skill dependent and reducing both the average insertion time and the insertion time variability from procedure-to-procedure. Potential benefits of the device disclosed herein include making the procedure less painful for the patient, reducing the risk of endoscope-related injuries in endoscopists and nurses, as well as reducing the procedure time and/or reducing/eliminating the need for sedation.

### SUMMARY

According to an embodiment of the disclosure, a deployment device is configured to guide an endoscope to an in vivo deployment location in a patient's colon. The deployment device comprises a flexible overtube having a distal end, a proximal end, a folded portion and an unfolded portion. An introducer is fixedly coupled to the proximal end of the flexible overtube, the introducer being adapted to be fixed in place adjacent a proximal end of the colon. A delivery element is coupled to the distal end of the flexible overtube, the delivery element including a steering element and a propulsion element wherein the steering element includes a proximal end and a distal end, wherein the distal end of the steering element includes a distal end of the deployment device when the deployment device is in use. The propulsion element is disposed adjacent the steering element and exerts an axial force on the steering element to drive the steering element distally relative to the introducer thereby progressively unfolding and deploying the flexible overtube until the distal end of the overtube reaches the deployment location. The deployment of the flexible overtube results in an enclosed tubular cavity encompassed by the flexible overtube, the tubular cavity extending within the patient's colon from the introducer to the deployment location and being configured to guide the endoscope to the deployment location.

The deployment device may also include a fixation member attached to the distal end of the flexible overtube to maintain the distal end of the flexible overtube at the deployment location. This fixation member may be a balloon that is inflated to frictionally engage an inner surface of the colon. The deployment device may also include a containment member enclosing the folded portion of the overtube and maintaining the folded portion of the flexible overtube in a folded configuration. The folded portion of the flexible overtube may comprise a fold pattern for packaging of cylindrical tubes that includes stable inextensional post-buckling patterns of thin-walled cylinders under axial compression or combined axial-torsional loading. The propulsion element may include a propulsion balloon and a propulsion balloon inflation lumen configured to supply pressurized fluid to the propulsion balloon to inflate the propulsion balloon. The inflated propulsion balloon may form a seal with an inner surface of the flexible overtube

The axial force exerted on the steering element by the propulsion element may result from pressurized fluid pumped into the enclosed tubular cavity exerting a pressure force on a proximal surface of the propulsion element. Further, the steering element may have a cylinder including a flexible material and two or more internal chambers, wherein each internal chamber may be selectively pressurized and depressurized, further wherein selective pressurization of the chambers results in the cylinder bending in a steering direction. A multi-lumen catheter may also be provided the supplies pressurized fluid to each internal chamber, the catheter extending to the introducer and a fluid supply system adapted to control the pressure in the chambers. The fluid supply system of any embodiment may be automated.

The deployment device may include an insufflation sealing member attached to the introducer, the insufflation sealing member configured to create a seal between the introducer and the patient's colon. Such device may also have a colon insufflation port adjacent to distally of the insufflation sealing member, the insufflation port configured to supply pressurized fluid to the patient's colon to insufflate the patient's colon. A fixation member may be attached to the distal end of the flexible overtube, wherein the fixation member is a balloon configured to maintain the distal end of the flexible overtube at the deployment location and further wherein the steering element is configured to receive the folded portion of the flexible overtube and the fixation balloon.

An embodiment provides a method for guiding an endoscope to an in vivo deployment location in a patient's colon. The method including inserting an introducer into a proximal end of the patient's colon and fixing the introducer in place adjacent the proximal end of the colon. A deployment device may be advanced distally into the patient's colon, the deployment device being connected to the introducer by a flexible overtube, wherein a distal end of the flexible overtube is coupled to the deployment device and a proximal end of the flexible overtube is coupled to the introducer. The deployment device may include a steering element and a propulsion element, the steering element having a proximal end and a distal end, the distal end of the steering element comprising a distal end of the deployment device when the deployment device is in use. The method may include deploying the flexible overtube, the flexible overtube having a folded portion and an unfolded portion and deploying the overtube may involve extending the unfolded portion by unfolding the folded portion until the distal end of the deployment device reaches the deployment location, wherein deploying the overtube results from advancing the deployment device and further wherein deploying the flexible overtube results in an enclosed tubular cavity encompassed by the flexible overtube. The method may also include supplying pressurized fluid to the enclosed tubular cavity of the overtube, the pressurized fluid exerting an axial force on the propulsion element of the deployment device and advancing the deployment device distally relative to the introducer. The method may also involve inserting the endoscope into the proximal end of the flexible overtube, through the enclosed tubular cavity to the distal end of the overtube and, thus, to the deployment location.

The method may also include inflating a balloon attached to the distal end of the flexible overtube, whereby the distal end of the flexible overtube frictionally engages an inner surface of the colon and the flexible overtube is fixed in place in relation to the patient's colon. Also, the steering element may be configured to receive the folded portion of the flexible overtube and the balloon prior to the inflation of the balloon.

The deployment device in this method may also include a deployment balloon and the method further comprising: inflating the propulsion balloon, wherein the axial force is exerted on a proximal portion of the inflated propulsion balloon; and creating a seal between the inflated propulsion balloon and an inner surface of the flexible overtube. The steering element may comprise a flexible cylinder enclosing two or more internal chambers and the method further comprising steering the deployment device by selectively pressurizing and depressurizing the chambers, resulting in the flexible cylinder bending in a steering direction. The method may also include creating a seal between the introducer and the patient's colon by inflating a sealing member attached to the introducer and insufflating the patient's colon by pumping pressurized fluid into the patient's colon distally of the insufflation sealing member through an insufflation port.

### BRIEF DESCRIPTION OF THE DRAWINGS

Further advantages and features of the present disclosure will become apparent from the following detailed description and the accompanying drawings.

FIGS. 1A through 1E illustrate examples of colonoscope looping in sigmoid colon and transverse colon.

FIG. 2A illustrates a perspective view an exemplary guiding assembly in accordance with various aspects of the disclosure.

FIG. 2B illustrates a cross sectional perspective view of the guiding assembly of FIG. 2A.

FIG. 2C illustrates a partial, cross sectional perspective detail view of the guiding assembly of FIG. 2A, with the fixation balloon in inflated state.

FIG. 2D illustrates a partial, cross sectional perspective detail view of the guiding assembly of FIG. 2A, with the fixation balloon in deflated state.

FIGS. 3A and 3B illustrate exemplary manufacturing methods of an overtube and a fixation balloon inflation channel of the guiding assembly of FIG. 2A.

FIG. 4 illustrates an exemplary manufacturing and assembly method of the fixation balloon of the guiding assembly of FIG. 2A.

FIG. 5A illustrates an exemplary origami fold pattern on a flat sheet representing an opened up cylindrical overtube of the guiding assembly.

FIG. 5B illustrates a side perspective view of the overtube of the guiding assembly of FIG. 2A in a partially collapsed, stored state.

FIG. 5C illustrates a cross-sectional perspective view of the partially collapsed overtube of FIG. 5B.

FIG. 6A illustrates a partially transparent, perspective view of an exemplary delivery assembly for use with the guiding assembly of FIG. 2A.

FIG. 6B illustrates a cross sectional perspective view of an exemplary delivery assembly of FIG. 6A.

FIG. 7 illustrates a cross sectional, perspective view of an exemplary catheter holder of the delivery assembly of FIG. 6A.

FIG. 8 illustrates an enlarged view of a steering mechanism and propulsion balloon of the delivery assembly of FIG. 6A.

FIG. 8B illustrates a simplified view of an exemplary steering mechanism of the delivery assembly of FIG. 6A.

FIG. 9 shows the cross section of an exemplary catheter.

FIG. 10 is a graph of an exemplary valve schedule for the three steering control valves that control the pressure in steering chambers of the steering mechanism of FIGS. 6A and 9.

FIG. 11 illustrates a cross sectional side view of the guiding assembly of FIG. 2A coupled to the delivery assembly of FIG. 6A.

FIG. 12 illustrates a partially transparent, cross sectional perspective view of the guiding assembly of FIG. 2A coupled with the delivery assembly of FIG. 6A.

FIG. 13 illustrates several cross sectional views of the guiding assembly at four states of deployment of the flexible overtube.

FIG. 14 illustrates a cross sectional side view of the guiding assembly of FIG. 2A in a partially deployed configuration.

FIG. 15A illustrates an exemplary origami folding pattern to be applied to fold a planar fixation balloon in a compact package.

FIGS. 15B and 15C are, respectively, a perspective view and side view of the fixation balloon, folded using the fold pattern of FIG. 15A, to encompass a distal end of the flexible overtube in collapsed orientation.

FIG. 16 is a perspective view of the folded fixation balloon of FIG. 15 disposed over the collapsed distal end of the partially deployed flexible overtube.

FIGS. 17A through 17F illustrate steps in an exemplary deployment and fixation of the deployment device into a colon, to facilitate straightforward insertion of the colonoscope through the guiding tube.

FIG. 18 is a schematic view of an exemplary fluid control system for use with the guiding assembly and the delivery assembly of FIG. 11.

FIG. 19A is an enlarged, perspective side view of the electromagnetic coil 248 shown in FIG. 11.

FIG. 19B is a perspective view of the electromagnetic coil tracking setup in accordance with an embodiment of this disclosure.

FIG. 20A illustrates a 2D representation of an exemplary display of device trajectory presented by the EM tracking setup in accordance with an embodiment of this disclosure.

FIG. 20B illustrates a 3D representation of an exemplary display of device trajectory presented by the EM tracking setup in accordance with an embodiment of this disclosure.

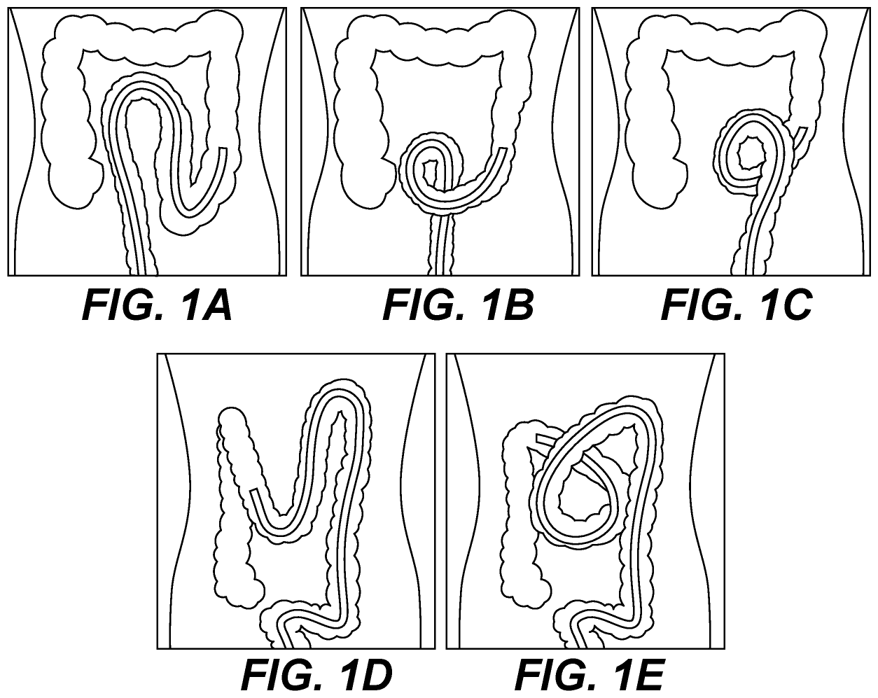

### DETAILED DESCRIPTION

Reference will now be made in detail to presently preferred embodiments and methods of the present disclosure, which constitute the best modes of practicing the present disclosure presently known to the inventors. The figures are not necessarily to scale. However, it is to be understood that the disclosed embodiments are merely exemplary of the present disclosure that may be embodied in various and alternative forms. Therefore, specific details disclosed herein are not to be interpreted as limiting, but merely as a representative basis for any aspect of the present disclosure and/or as a representative basis for teaching one skilled in the art to variously employ the present disclosure. Furthermore, the terminology used herein is used only for the purpose of describing particular embodiments of the present disclosure and is not intended to be limiting in any way.

As used in the specification and the appended claims, the singular form “a.” “an,” and “the” include plural referents unless the context clearly indicates otherwise. For example, reference to a component in the singular is intended to also include a plurality of components.

Also, as used in the specification and the appended claims, the terms “distal” and “proximal” are defined relative to the device/endoscopist and not relative to the patient/colon. Thus, for example, a “distal” element is relatively further away from the endoscopist than a “proximal” element, which is relatively closer to the endoscopist. The terms “distal” and “proximal” apply to references to the device as well as the colon. As used in the specification and the appended claims, “proximal” colon refers to the anus and “distal” colon refers to the caecum, which is reverse of medical terminology.

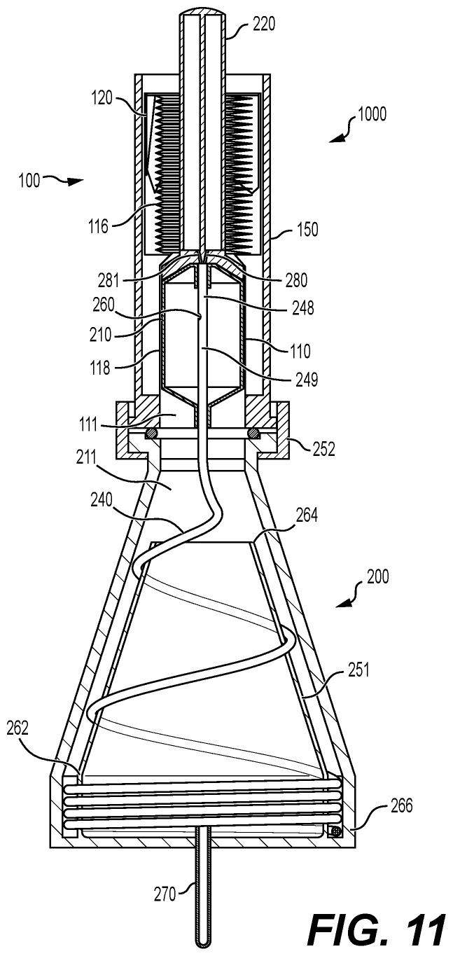

Referring to FIG. 11, a deployment device 1000 is configured to receive and guide an endoscope to an in vivo deployment location. The primary components of deployment device 1000 include the guiding assembly 100 and the delivery assembly 200, each of which will be described in greater detail below. The guiding assembly 100 will be disposed in the colon of the patient. The guiding assembly contains an introducer 150 which will be disposed adjacent to the anus of the patient and may engage the anus. The delivery assembly 200 contains a multi-lumen catheter 240 that may be fed from the catheter holder 250 into the colon. These components may be removably coupled together such that the introducer 150 can support the guiding assembly 100 and operate as a connection between the guiding assembly 100 and the delivery assembly 200.

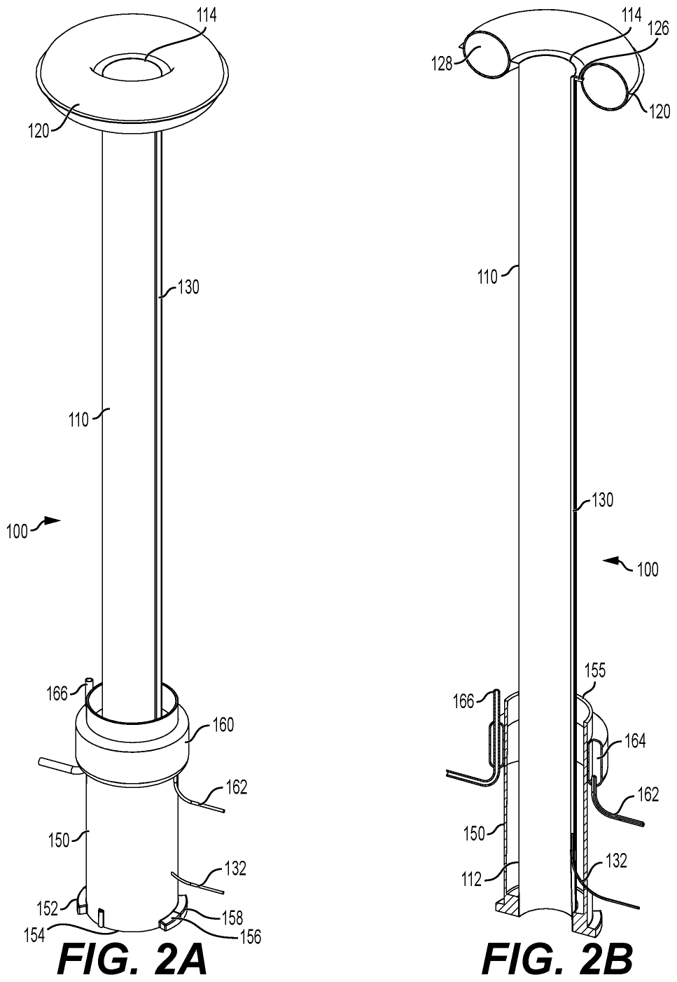

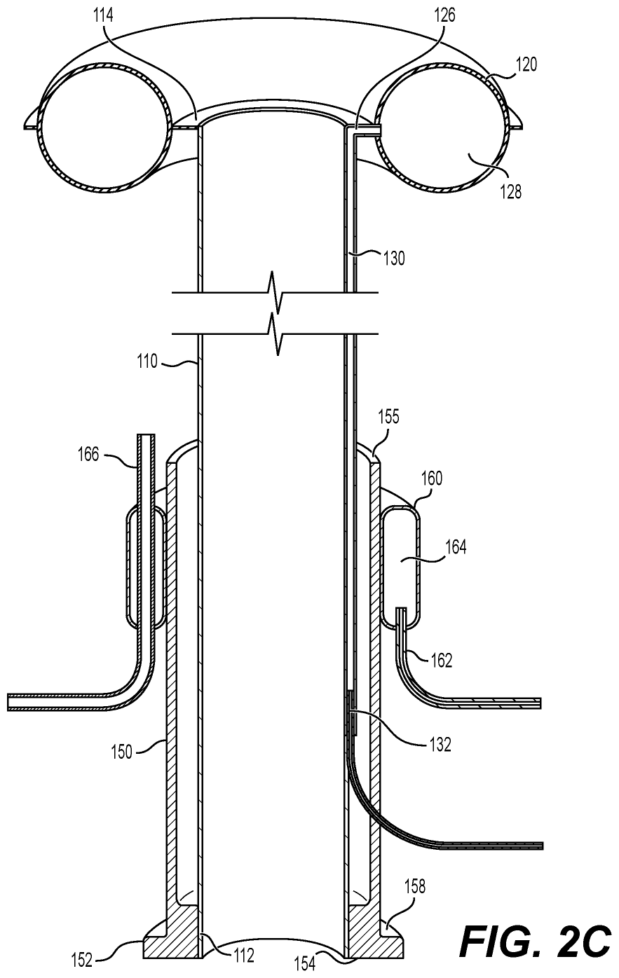

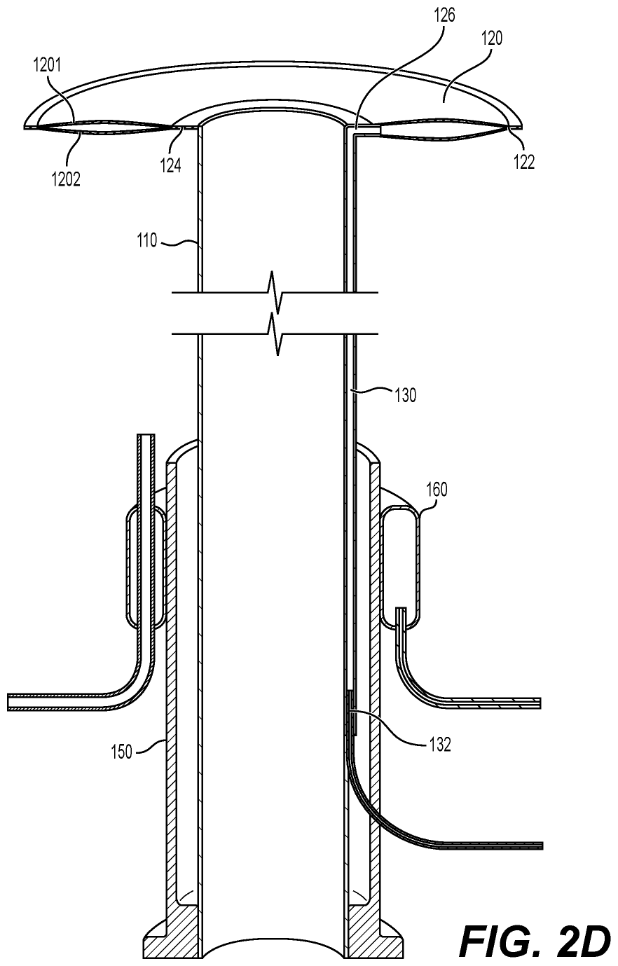

FIGS. 2A-2C illustrate an exemplary guiding assembly 100 in accordance with various aspects of the disclosure. In the illustrated embodiment, the guiding assembly 100 includes a flexible overtube 110 and an introducer 150 attached at a proximal end 112 of the flexible overtube 110.

The flexible overtube 110 may include a toroidal/donut-shaped fixation balloon 120 at a distal end 114. A fixation balloon inflation channel 130 is incorporated alongside the flexible overtube 110, extending from the proximal end 112 to the distal end 114. The fixation balloon inflation channel 130 is in fluid communication with the fixation balloon cavity 128 by means of opening 126 to allow for inflation and deflation of the fixation balloon 120, and is connected to a fixation balloon inflation port 132 adjacent the proximal end 112.

In some aspects, the flexible overtube 110 or the fixation balloon 120 comprise thermoplastic polyurethane (TPU), thermoplastic elastomer (TPE), nylon, polyether block amide (PEBA), polyethylene (PE), or silicones. While the flexible overtube 110 is made from flexible, compliant material, the material of the overtube 110 should not be amenable to significant stretch. The flexible overtube 110 may be easily bent, folded, unfolded or deployed, as it is made from low thickness material. In some embodiments, the thickness of the material may be in the range of 0.01 mm-0.75 mm. That is, the material of the flexible overtube 110 should not expand significantly under biaxial, radial or circumferential forces on the overtube material. Once the overtube 110 is deployed, the overtube material should not expand to a significant degree, either radially or axially. That is, the deployed overtube 110 does not stretch significantly like a balloon when the pressure inside the overtube 110 is increased. In some aspects, the introducer 150 comprises a rigid plastic material such as polypropylene (PP), polyvinyl chloride (PVC), polystyrene, nylon, polycarbonate (PC), polyethylene terephthalate (PET), acrylonitrile butadiene styrene (ABS) or polymethacrylate. In some embodiments, the flexible overtube 110 may be approximately 3 feet long. However, it should be appreciated that the flexible overtube 110 could have a shorter length or a longer length, depending on the use case.

FIG. 2D shows the fixation balloon 120 in a flat and deflated state. After deployment, the fixation balloon 120 of the guiding assembly 100 is inflated and, as shown in FIGS. 2A, 2B and 2C, assumes a toroidal/donut shape to fix the distal end 114 of the guiding assembly 100 in the colon. As discussed further hereinbelow, upon deployment and fixation of the distal end 114 of the guiding assembly 100, the delivery assembly 200 may be removed from the deployed and fixated guiding assembly 100.

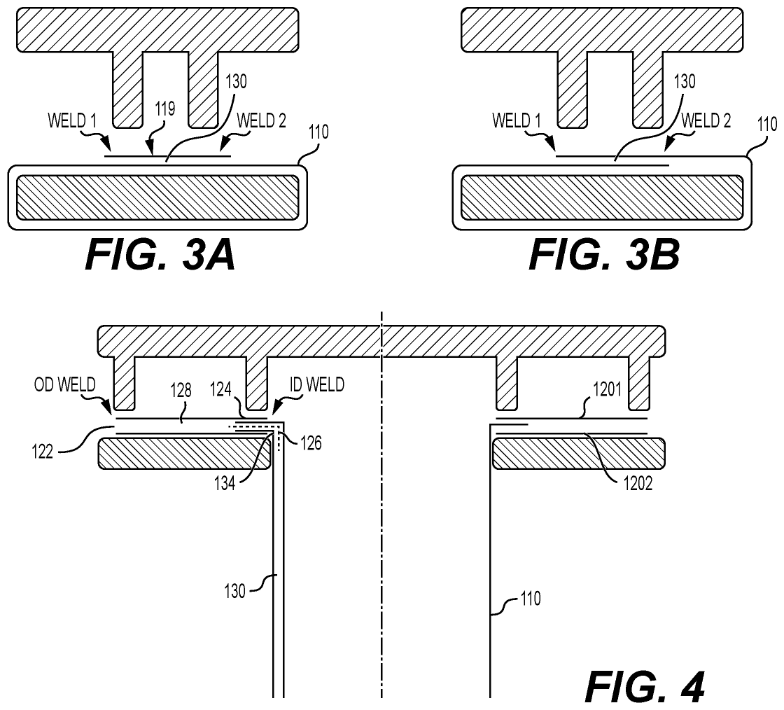

As shown in FIG. 3A, in some embodiments, the flexible overtube 110 may be made using blown film extrusion or another method, and an additional elongated rectangular TPU sheet 119 may be attached, for example, by welding, to the flexible overtube 110 by means of two parallel welds to create the attached fixation balloon inflation channel 130. As shown in FIG. 3B, in some embodiments, the flexible overtube 100 may be made by wrapping a flat rectangular sheet of TPU around a flat (or shaped) mandrel, and welding the two ends of the sheet by means of two parallel straight-line welds to simultaneously create the flexible overtube 110 with the fixation balloon inflation channel 130 alongside the flexible overtube 110. In another exemplary method, the flexible overtube 110 and the fixation balloon inflation channel 130 may be made by a continuous process using a tube former and making two parallel welds. Welding may be achieved by means of thermal welding or RF (radio-frequency) welding. In another embodiment, adhesives or other joining techniques may be used for attachment instead of welding.

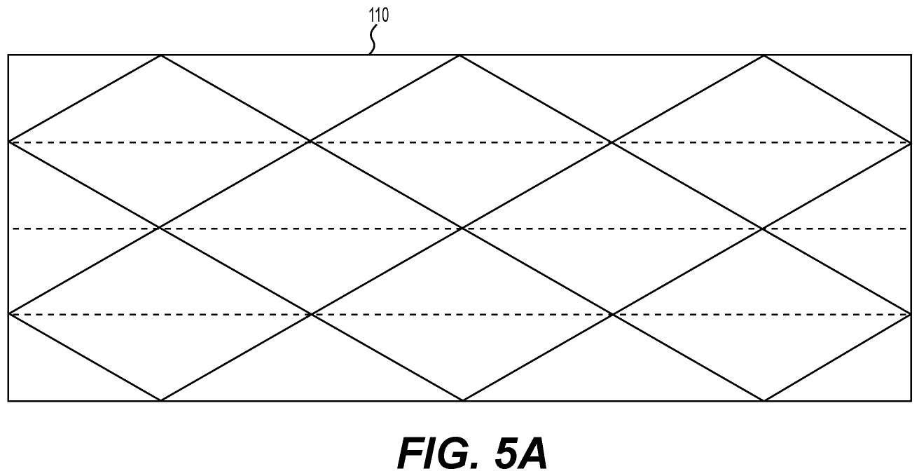

Referring now to FIG. 4, the fixation balloon 120 may be made by welding two flat, circular sheets 1201, 1202 of polymer material. e.g., TPU, at the inner diameter 124, followed by welding at the outer diameter 122. At the same time as the welding of the inner diameter 124 of the fixation balloon 120, the flexible overtube 110 and the fixation balloon inflation channel 130 may be simultaneously attached to the fixation balloon 120 at the inner diameter 124. A tape 134, for example, a Teflon tape, may be placed within the fixation balloon inflation channel 130 during welding of the inner diameter 124 to maintain a fluid passage 126 between the fixation balloon cavity 128 and the fixation balloon inflation channel 130. After completion of the weld at the inner diameter 124, the tape 134 is removed to create opening 126. This is followed by welding of the outer diameter 122 to create the fixation balloon cavity 128. The opening 126 permits fluid communication between the fixation balloon cavity 128 and the fixation balloon inflation channel 130. Welding may be achieved by means of thermal welding or RF (radio-frequency) welding. In another embodiment, adhesives or other joining techniques may be used for attachment instead of welding. In another embodiment, the fixation balloon may be directly formed in a toroidal/donut shape.

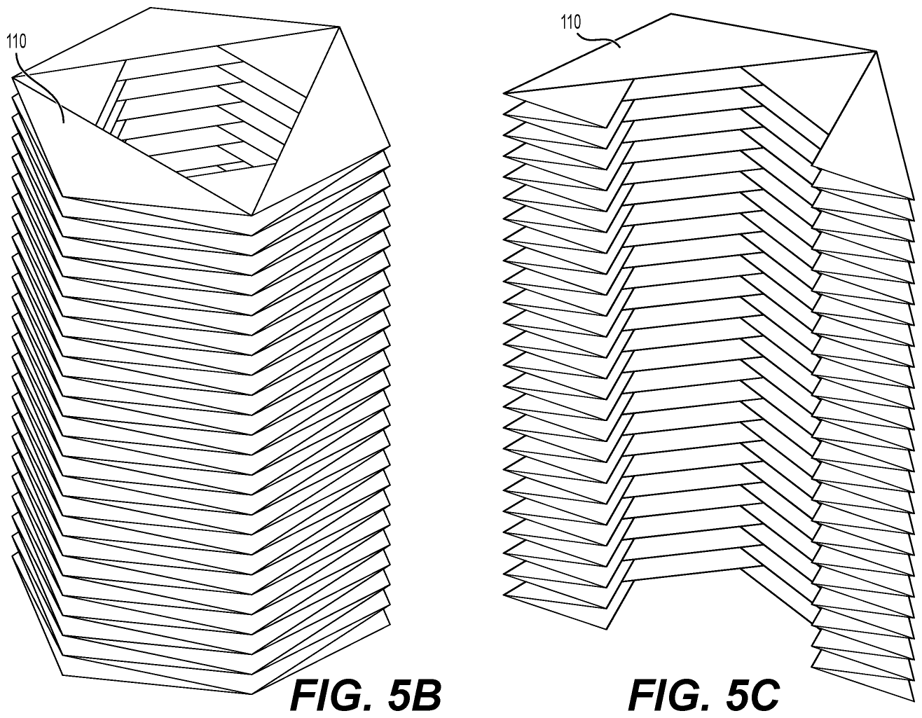

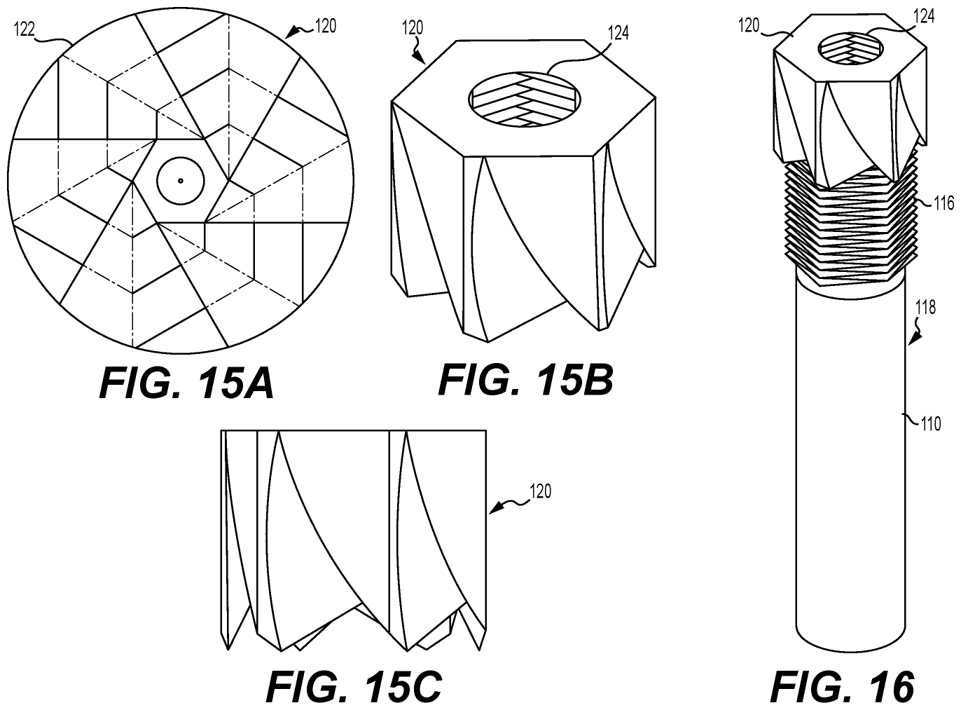

FIG. 2A illustrates the flexible overtube 110 and fixation balloon 120 of the guiding assembly 100 in an extended or deployed state. FIGS. 5B, 5C. 11.12, and 16 show the flexible overtube 110 packaged into a smaller axial length. FIGS. 11.12, 15B, 15C and 16 also illustrate the fixation balloon 120 in an uninflated and folded state. A folding process is described in greater detail hereinbelow, resulting in the flexible overtube 110 and fixation balloon 120 occupying a smaller profile shown in FIGS. 5B, 5C, 15B. 15C and 16 for insertion into the anus and deployment into the colon.

As shown in FIG. 16, in a partially packaged state, a folded (or packaged) portion 116 of the flexible overtube 110 remains axially packaged, i.e., axially compressed or folded, and an unfolded (or expanded) portion 118 of the flexible overtube 110 is in the expanded or deployed state, i.e., not compressed or folded. As the distal end 114 of the guiding assembly 100 proceeds further into the patient's colon, more of the folded portion 116 of the overtube 110 is unfolded to form the unfolded (or expanded) portion 118. Put another way, as the flexible overtube 110 is disposed into the colon, the folded portion 116 progressively decreases in length while the unfolded (or expanded) portion 118 of the flexible overtube 110 progressively increases in length. FIG. 16 also illustrates the fixation balloon 120 folded into a smaller diameter profile.

By way of example, in some aspects, efficient packaging of one or both of the flexible overtube 110 and the fixation balloon 120 can be achieved by a number of origami-based folding techniques. Origami-based folding techniques have been developed and employed, for example, for compact packaging of deployable space structures within the packaging constraints of a rocket envelope to allow efficient transportation to space. Three such origami folding patterns for the flexible overtube 110 include (a) the Yoshimura pattern, (b) the bellows fold pattern, and (c) helically triangulated folds. A person skilled in the art will know a wide range of origami patterns that can be used for folding cylinders and flat membranes. Efficient axial packaging of the flexible overtube 110 can be achieved by means of various patterns derived from stable inextensional post-buckling patterns of thin-walled cylinders under axial compression or under combined axial and torsional loading. Examples are disclosed in Hunt, G. W., and Ario, I., “Twist Buckling and the Foldable Cylinder: An Exercise in Origami”, International Journal of Non-Linear Mechanics, Vol. 40, No. 6, pp. 833-43 (2005); Schenk et. al., “Review of Inflatable Booms for Deployable Space Structures: Packing and Rigidization”. Journal of Spacecraft and Rockets, Vol. 51, No. 3 (May-June 2014); and Johnson, W., Soden, P. D., and Al-Hiassani, S. T. S., “Inextensional Collapse of Thin-Walled Tubes Under Axial Compression”, Journal of Strain Analysis for Engineering Design, Vol. 12, No. 4, pp. 317-30 (1977). Each of these references is incorporated herein by reference in its entirety.

FIG. 5A shows the Yoshimura pattern, which is one example of a fold pattern that may be used for packaging the flexible overtube 110. FIG. 5A shows the fold pattern on a flat sheet formed if the cylindrical flexible overtube 110 were cut along the length of the cylinder. The solid lines represent mountain folds and dotted lines represent valley folds. Folding the flexible overtube 110 utilizing the fold pattern of FIG. 5A results in the folded, collapsed overtube shown in FIG. 5B and in cross-section in FIG. 5C.

For the fixation balloon 120 in the flattened, deflated state, a modified NASA Star-Shade origami pattern with a hexagonal base, octagonal base or another suitable base may be employed. FIG. 15A illustrates an example star-shade origami pattern that may be used for folding and packaging the flattened, deflated fixation balloon 120. The solid lines of FIG. 15A represent mountain folds and the dotted lines represent valley folds. FIGS. 15B and 15C show the resulting folded fixation balloon 120. FIG. 16 shows the fixation balloon 120 of FIGS. 15B and 15C folded over the distal end of the folded portion 116 of the flexible overtube 110. It is also to be remembered that the fixation balloon 120 is attached to the distal end 114 of the flexible overtube 110 at the inside diameter 124 of the fixation balloon 120.

The modified NASA Star-Shade origami fold pattern with hexagonal base for the fixation balloon 120 is compatible with the Yoshimura fold pattern used for the flexible overtube 110, to enable efficient packaging of the fixation balloon 120 around the flexible overtube 110.

A person skilled in the art will know a wide range of origami patterns that can be used for folding cylinders and flat membranes. A summary of several origami folding patterns can be found in Nojima T. Modelling of Folding Patterns in Flat Membranes and Cylinders by Origami. JSME International Journal Series C. 2002; 45(1):364-370. This reference is incorporated herein by reference in its entirety.

In some aspects, efficient packaging of the flexible overtube 110 and the fixation balloon 120 can be achieved by means other than folding or origami folding of the overtube 110 or the fixation balloon 120. In some aspects, the overtube 110 may be shortened by axially bunching up or crumpling it in the axial direction. Similarly, the fixation balloon 120 can be folded over the distal end of the shortened flexible overtube 110 without a pattern and without origami techniques.

The fixation element need not be a fixation balloon. It alternate embodiments, the fixation element may be a structure that is configured packaged around the folded portion of the flexible overtube during deployment, and configured to be expanded for fixation at the deployment location in the colon.

Referring again to FIGS. 2A, 2B and 2C, the introducer 150 includes a circumferentially discontinuous connection portion 152 adjacent the proximal end 154. The circumferentially discontinuous connection portion 152 may include a lug or holding surface 158 and an inclined surface 156 to permit attachment and detachment of a delivery assembly 200, which is described in more detail below, to the introducer 150. The guiding assembly 100 can include an insufflation sealing balloon 160 that may be disposed about the introducer 150 at a distal end 155 of the introducer, and an insufflation sealing balloon port 162 in fluid communication with a cavity 164 of the insufflation sealing balloon 160. The insufflation sealing balloon 160 may also be referred to as a sealing cuff. The insufflation sealing balloon 160 may be a single, donut shaped balloon. In some embodiments, it may be formed from two concentric tube segments that are welded or attached at two axial locations. In yet another embodiment, it may be formed by attaching one flexible tube to the rigid introducer 150 at two axial locations. Either way, insertion of the introducer 150 into the anus is performed with the insufflation balloon 160 deflated. Inflation of the balloon 160 creates a mechanical engagement with the patient's anus and the introducer 150 is fixedly held in place. Deflation of the insufflation sealing balloon 160 permits either adjustment or removal of the introducer 150. Any structures physically connected to the introducer 150 will also be maintained in place along with the introducer 150.

Another function of the insufflation sealing balloon 160 is to physically seal the lumen of the patient's colon from the atmosphere outside the patient's body. Once the introducer 150 and structures attached thereto are fixedly held in place, e.g., by the sealing cuff 160, it is often beneficial to pressurize the colon of a patient with air, carbon dioxide, water or another fluid. The seal provided by the insufflation sealing balloon 160, serves to retain this fluid pressure in the colon lumen. Thus, guiding assembly 100 may include a colon insufflation port 166 configured to insufflate the colon to a prescribed pressure with, for example, air or carbon dioxide or water. In the illustrated embodiment, the colon insufflation port 166 is routed to the colon through the insufflation sealing balloon 160. The colon insufflation portion 166 can be routed through or parallel to another portion of the introducer 150, as long as the pressurized fluid from outside the colon to a portion to the colon ‘sealed’ from the atmosphere, e.g., by the insufflation sealing balloon 160. In another embodiment, the sealing cuff 160 may consists of two axially spaced donut shaped balloons about the introducer 150 to achieve more secure attachment at the anus and better sealing of the colon insufflation pressure.

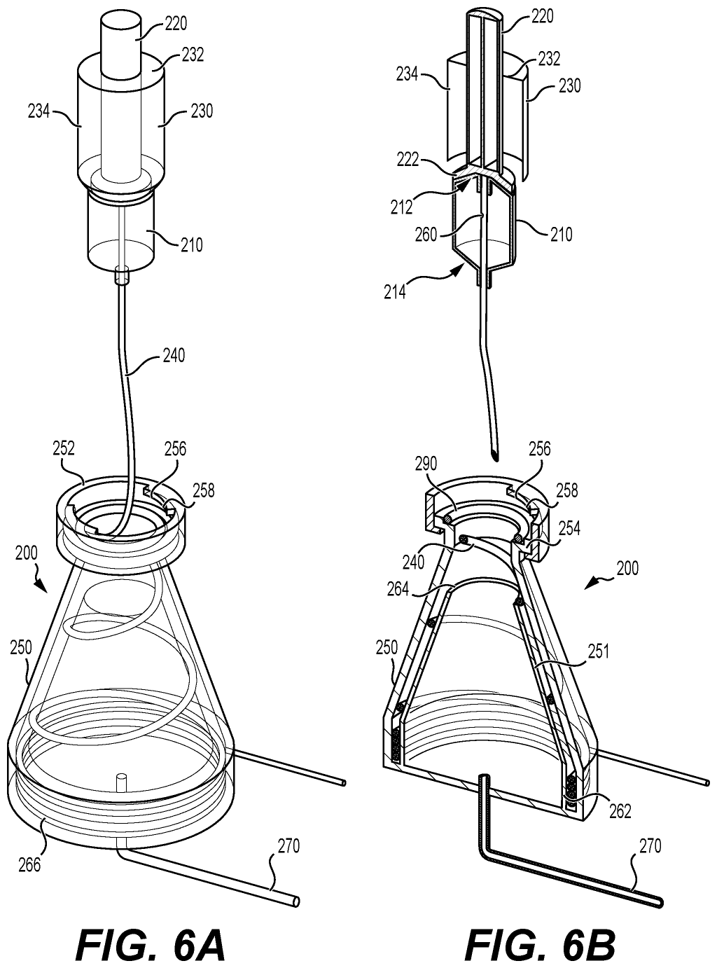

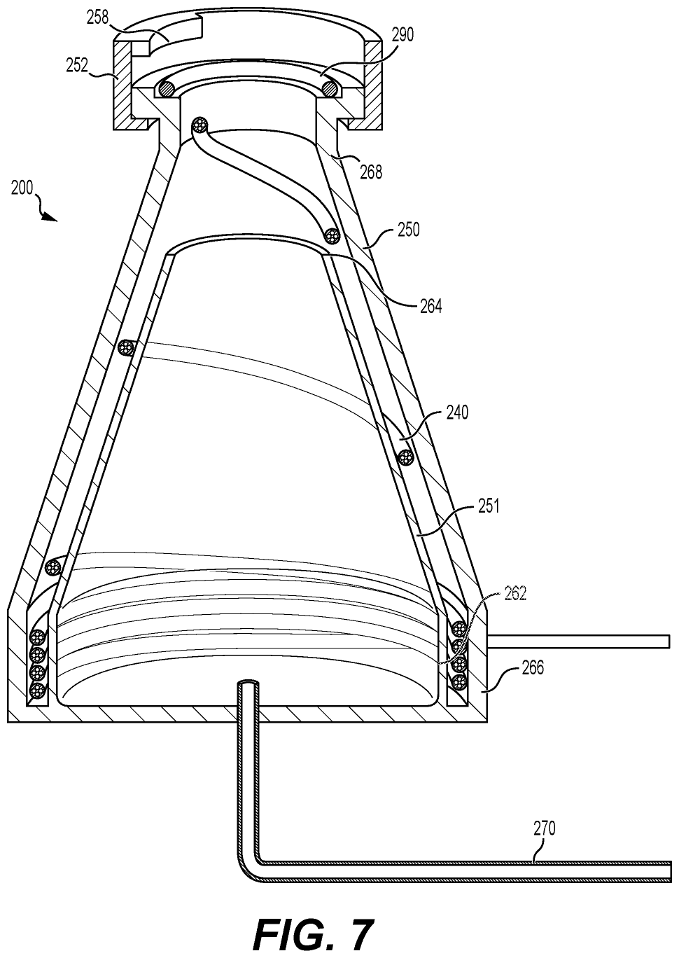

Referring now to FIGS. 6A, 6B and 7, an exemplary delivery assembly 200 in accordance with various aspects of the disclosure is illustrated. As noted, the delivery assembly 200 may be connected to the guiding assembly 100 by way of the introducer 150.

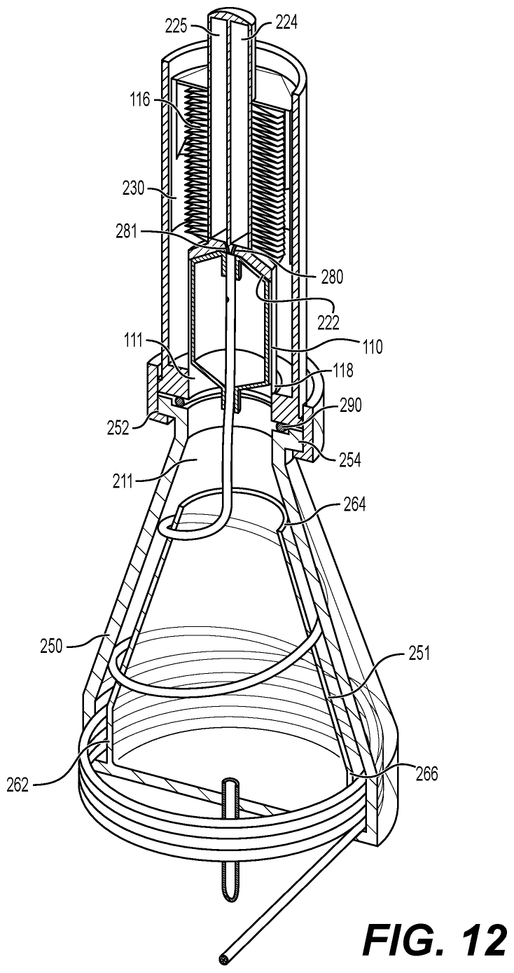

The delivery assembly 200 includes a propulsion balloon 210, a steering mechanism 220, a fold containment member 230, a multi-lumen catheter 240, and a catheter holder 250. Note that the distal end of the delivery assembly 200 has components that are closely engaged with elements of the guiding assembly 100. For example, the steering mechanism 220 extends through the folded portion 116 of the flexible overtube 110 of the guiding assembly 100; the fold containment member 230 surrounds the folded portion 116 of the flexible overtube 110 as well as the fixation balloon 120; and the propulsion balloon 210 abuts both the folded portion 116 and the expanded portion 118 of the flexible overtube 110, as best seen in FIGS. 11 and 12.

The propulsion balloon 210 is one example of a propulsion element for use in the delivery assembly 200. Features of the propulsion element are that it forms and seals a flexible overtube cavity 111 in cooperation with the unfolded portion 118 of the flexible overtube 110; transmits a distally directed axial force to the steering mechanism 220 to displace the steering mechanism 220 through the colon; and provides a mechanism to unfold the folded portion 16 of the flexible overtube 110 progressively from the proximal end to the distal end. To be clear, the propulsion element need not be a balloon. The propulsion element 210 may create a perfect seal for the overtube cavity 111, or may create a leaky seal for the overtube cavity 111.

The collection of elements that includes some or all of the propulsion element 210, flexible overtube 110, steering mechanism 220 and fold containment member 230, as well as additional structural elements, may be referred to as the propulsion assembly. Put another way, the propulsion assembly is that portion of the device that is propelled distally in the colon or other body lumen.

In some embodiments, deliberate steering functionality may not be required. In such embodiments, the steering mechanism 220 may be a passive cylinder and not provide any steering action. The cylinder may be flexible or rigid, and may be hollow or filled. In such embodiments, where no steering functionality is required or provided, the passive cylinder 220 along with the base 222 and the radially extending fold containment portion 232 may together serve as a fold containment element.

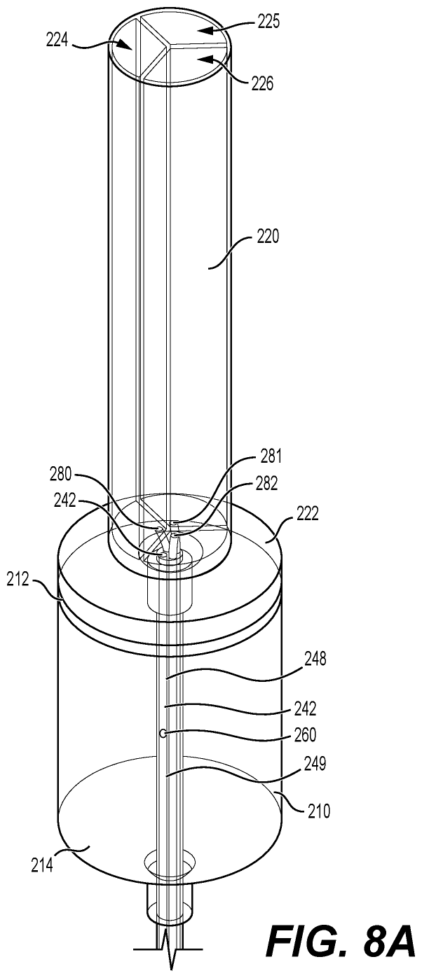

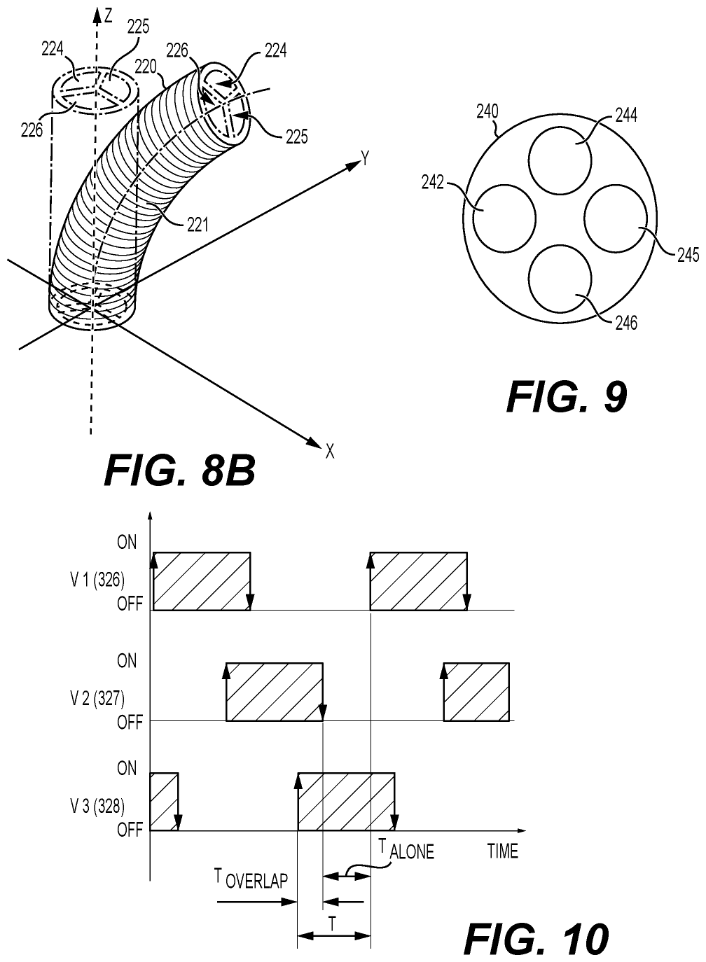

As best shown in FIG. 6B and FIG. 8A, a base 222 of the steering mechanism 220 may contact a distal end 212 of the propulsion balloon 210. In the illustrated embodiment, the base 222 of the steering mechanism 220 has a conical profile. However, it should be understood that, in other embodiments, the base 222 may have a flat profile or any other profile as long as it enables progressive unfolding of the folded portion 116 of the flexible overtube 110 from the proximal end 112 to distal end 114 as described in detail later.

The propulsion balloon 210 may be substantially cylindrical or elliptical (instead of spherical) in shape. This can be characterized by a L:D ratio of effective length (L) to diameter (D) of the propulsion balloon. A person skilled in the art would understand that a spherical balloon with a line contact with the flexible overtube 110 will limit the bending moment that can be transmitted from the steering actuator to the flexible overtube 110. An inflated cylindrical or elliptical propulsion balloon 210 within the flexible overtube 110 has the capability to transmit greater forces and bending moments. The L:D ratio should not be too high since a higher L:D ratio may increase the frictional force between the propulsion balloon 210 and the flexible overtube 110 and may also increase the turning radius for the flexible overtube 110. In some embodiments, L:D ratios between about 0.75:1 up to 2:1 or about 0.5:1 up to 5:1 have been shown to be useful.

In the preferred embodiment, as shown in FIG. 8A and simplified FIG. 8B, steering mechanism 220 may include a thin-walled flexible silicone cylindrical actuator, with three symmetric internal chambers 224, 225 and 226, and circumferentially oriented reinforcing-fibers 221. The circumferentially oriented reinforcing fibers 221, shown in simplified FIG. 8B, prevent or minimize radial expansion of the cylindrical actuator, but allow for elongation of individual chambers. When the internal pressure in the three chambers 224, 225, 226 is increased equally, the steering actuator may stretch in the axial direction. When the pressure of one or two of the three chambers 224, 225, 226 is increased, the steering actuator bends in a direction opposite the pressurized chamber(s). For instance, a simplified representation of the steering mechanism 220 in FIG. 8B shows that increased fluid pressure supplied to steering chambers 224 results in the steering mechanism 220 bending in the direction away from the pressurized chamber 224. It should be understood that the FMA can be bent in a desired direction by independently controlling the pressures in the three chambers 224, 225 and 226, to achieve movement with three degrees of freedom (pitch, yaw, and stretch). Static and dynamic deformation characteristics of the steering actuator are described in the publications: Suzumori K. Flexible microactuator (1st Report, Static characteristics of 3 DOF actuator). Transactions of the Japan society of Mechanical Engineers Series C. 1989; 55(518):2547-2552; and Suzumori K. Flexible microactuator (2nd Report. Dynamic characteristics of 3 DOF actuator). Transactions of the Japan society of Mechanical Engineers Series C. 1990; 56(527):1887-1893. Each of these references is incorporated herein by reference in its entirety.

The steering mechanism 220 can be made out of silicone or another polymer/rubber of high flexibility and stretchability. Alternately, it could be made out of thermoplastic elastomers or thermoplastic polyurethane or thermoplastics vulcanizate or another material of high flexibility and stretchability. In one embodiment, there may be an axial separation between the three chambers 224, 225, 226 (the hollow portion) and the base 222 of the steering device 220. That is, the chambers may occupy only a portion of the length of the steering device 220, e.g., only the top two-thirds or one-half of the device 220.

The steering element may be another shape instead of a cylinder, and may include two or more chambers. In one embodiment, the steering element may include three cylindrical chambers resulting in a different shape for the overall steering element. In some embodiments, the steering element may not include fiber reinforcement.

In some other embodiments, the steering element may include a different operating principle instead of pneumatic or hydraulic actuation. The steering element may be an electric motor driving a shaft with an off-axis weight; it may be a vibrating motor; it may include Bowden-cable pull-wires commonly used in catheter steering; it may include multiple fluid jets at the distal end oriented in radial, axial or circumferential orientation to apply forces or torque on the distal end to steer the distal end of the flexible overtube; or it may include another steering mechanism known to those skilled in the art.

Catheter 240 may include one or more lumens as shown in the cross-section view in FIG. 9. One such lumen may be a propulsion balloon inflation lumen 242 that is in fluid communication with the propulsion balloon 210 by means of propulsion balloon inflation port 260 in the catheter 240, as shown in FIGS. 6B and 8A. The propulsion balloon inflation lumen may be used to inflate and/or deflate the propulsion balloon 210.

In addition to the propulsion balloon inflation lumen 242, the catheter 240 may comprise multiple lumens, for example three steering pressure lumen 244, 245 and 246, as shown in FIG. 9. The steering pressure lumen 244, 245 and 246 are respectively in fluid communication with steering chambers 224, 225 and 226 of the steering mechanism 220, through the steering ports 280, 281 and 282, as shown in FIG. 8A. The multi-lumen catheter 240 may be routed through the propulsion element, e.g., propulsion balloon 210, to establish a fluid connection between the steering chambers 224, 225, 226 of the steering mechanism 220 and a fluid control system 300, described in more detail below. It should be noted that the propulsion balloon inflation lumen 242 may be sealed at the distal end of the catheter.

In the illustrated embodiment, the multi-lumen catheter 240 may be stored in the catheter holder 250. The catheter 240 may provide fluid or fluid pressure to the propulsion balloon 210 and steering mechanism 220 of the delivery assembly 200 as they travel into the colon while the proximal end of the delivery assembly 200 remains attached to the anus or the fluid control system 300.

The catheter holder 250 includes a rotatable connection portion 252 adjacent a distal end 254 of the catheter holder. The connection portion 252 comprises a circumferentially discontinuous structure that includes an inclined surface 256 and a holding surface 258 (or lug 258) to permit attachment and detachment of the introducer 150 of the guiding assembly 100. The catheter 240 may be wound around a larger diameter winding cylinder 262 close to the proximal end 266 of the catheter holder 250, as shown in FIGS. 6A, 6B, 7, 11 and 12. The catheter 240 advances into the guiding assembly 100 from the distal end 268 of the catheter holder. The advancement is accompanied by twisting of the catheter and the number of twists is equal to the number of windings of the catheter 240 in the catheter holder 250. The larger diameter of the winding cylinder 262 reduces the number of windings required to accommodate the length of the catheter 240 and thus limits twisting of the catheter 240 upon deployment of the delivery assembly 200. The diameter 264 of the catheter holder at the distal end of the catheter holder is close to the diameter of the flexible overtube 110 of the guiding assembly 100. The catheter holder 250 may include a support portion 251, for example, a conical support portion, that is configured to provide a guiding surface for the catheter 240 to transition from the larger diameter winding cylinder 262 at the proximal end 266 to the smaller diameter 264 at distal end 268.

In another embodiment, the catheter may be stored outside the “catheter-holder” and may be fed into the flexible overtube cavity 111 as the propulsion balloon 210 and steering actuator 220 of the delivery assembly 200 advance into the colon. However, this may result in a leakage path for the propulsion pressure (Pₚᵣₒₚᵤₗₛᵢₒₙ), and may introduce noise from the leakage. Efforts at minimizing the leakage may introduce friction which will make the advancement of the propulsion balloon 210 and steering actuator 220 of the delivery assembly 200 more challenging.

The fold containment member 230 may be attached to the steering mechanism 220 at a location spaced distally from the base 222 of the steering mechanism 220. The fold containment member 230 is configured to contain the folded portion 116 of the flexible overtube 110 between itself and the base 222 of the steering device 220. The fold containment member 230 may include a radially extending containment portion 232. In some embodiments, the radially extending portion 232 may be circumferentially discontinuous. In some embodiments, the fold containment member 230 is configured to contain the folded portion 116 of the flexible overtube 110 and the folded fixation balloon 120. For example, the fold containment member 230 may include an axially extending containment portion 234 that is spaced from and, thus, does not interfere with operation of the steering mechanism 220. In some embodiments, the axially extending portion 234 may extend entirely or partially to the base of the steering actuator 222. In some embodiments, the axially extending portion 234 may be circumferentially discontinuous. The fold containment device 230 and the exposed portion of the steering mechanism 220 are the parts of the device in some embodiments that make contact with and move relative to the colon during the overtube 110 deployment. It may, therefore, be advantageous to have a friction reducing coating on these surfaces of the fold containment device and the exposed portion of the steering mechanism.

Those skilled in the art will recognize that the containment element may not use a mechanical containment mechanism. In some embodiments, fold containment may be achieved by use of fugitive glue (also known as temporary glue) or use of velcro, both of which get released upon deployment. In some embodiments, the folds in folded portion 116 of the flexible overtube 110 and the folded fixation balloon 120 may be set by thermally annealing the folded structure. In such embodiments, the thermally set folded shape may provide fold containment without the need for a containment member 230.

In the included figures, the containment device 230 is shown as having a “bluff-body” profile or a “non-aerodynamic” profile. In alternative embodiments, the profile of the containment device 230 may possess an “aerodynamic or sleek” profile to facilitate easier insertion into the colon. In the included figures, the steering mechanism 220 extends/protrudes axially beyond the distal end of the containment device. In an alternate embodiment, the containment device 230 may be connected to the steering device 220 at the distal tip. In some embodiments, the fold containment member 230 may have a low thickness and may be made out of flexible material (such as silicone, another rubber or thermoplastic elastomers or thermoplastic polyurethane or thermoplastics vulcanizate or another material of high flexibility and stretchability), such that the fold containment member 230 may invert inside-out to allow for easy removal of the delivery assembly 200 from the guiding assembly 100, after deployment and fixation of the distal end of the guiding assembly 110.

The deployment device 1000 includes the guiding assembly 100 and the delivery assembly 200 coupled together by means of the circumferentially discontinuous connection portion 152 of the introducer 150 and the circumferentially discontinuous connection portion 252 of the catheter holder 250. To couple the guiding assembly 100 and the delivery assembly 200, the circumferentially discontinuous connection portion 152 of the introducer 150 is axially inserted into the rotatable circumferentially discontinuous connection portion 252 of the delivery assembly 200. The connection portion is then rotated, causing the inclined surfaces 156 and 256 on the connection portions 152 and 252 to clamp the two assemblies 100, 200 together and to compress an O-ring 290 to seal the interface of the two assemblies 100, 200. The two assemblies 100, 200 are held together by the corresponding holding surfaces 158, 258 on the two connection portions 152, 252. Connection of the guiding assembly and the delivery assembly connects the delivery assembly cavity 211 with the flexible overtube cavity 111. Compression of the O-ring ensures that there is no leakage at that interface where the two cavities 111 and 211 are connected. It should be appreciated that other conventional mechanisms can be employed for removably connecting and sealing the two assemblies 100, 200.

In some embodiments, the guiding assembly 100 and the delivery assembly 200 may be fixedly coupled to one another, obviating the need for connectors 152 and 252 and O-ring 290.

As illustrated, e.g., in FIGS. 11 and 12, the flexible overtube 110 of the guiding assembly 100 may have a folded or packaged portion 116 and an unfolded or expanded portion 118. The folded portion 116 may be disposed distally of the unfolded portion 118. The fixation balloon 120 is folded around the folded portion 116 of the flexible overtube 110. The propulsion balloon 210 of delivery assembly 200 may be disposed within the unfolded portion 118 of the flexible overtube 110 of the guiding assembly 100. The folded portion 116 of the flexible overtube 110 and the folded fixation balloon 120 may be packaged distal to the propulsion balloon 210. The folded portion 116 of the flexible overtube 110 and the folded fixation balloon 120 may be disposed around the steering mechanism 220 and contained within the fold containment member 230.

During operation of the deployment device 1000 with the guiding assembly 100 and the delivery assembly 200 in an assembled state, the distal end of the deployment device 1000 is inserted through the anus and into the colon until the introducer 150 is adjacent the anus such that the flexible overtube 110, among other structures distal to the introducer 150, is deployed into the colon. In particular, placement of the sealing balloon 160 adjacent the anus is optimum placement in an embodiment of the device.

Normally, an empty and collapsed human colon presents challenging terrain for insertion of anything, including a flexible overtube 110. Since an insufflated colon, i.e., gas or other fluid filled colon, presents a more favorable terrain for insertion of the flexible overtube 110 compared to a collapsed colon, the colon may be insufflated prior to deployment of the flexible overtube 110 in some embodiments. Subsequent to placement of the introducer 150 adjacent the anus, the colon insufflation balloon 160 may be inflated to create a seal at the anus. The colon may be insufflated with carbon dioxide or air or water through the colon insufflation port 166 which is connected to an insufflation pressure (P_{Insufflation}) regulator of a fluid control system 300, as discussed in more detail below.

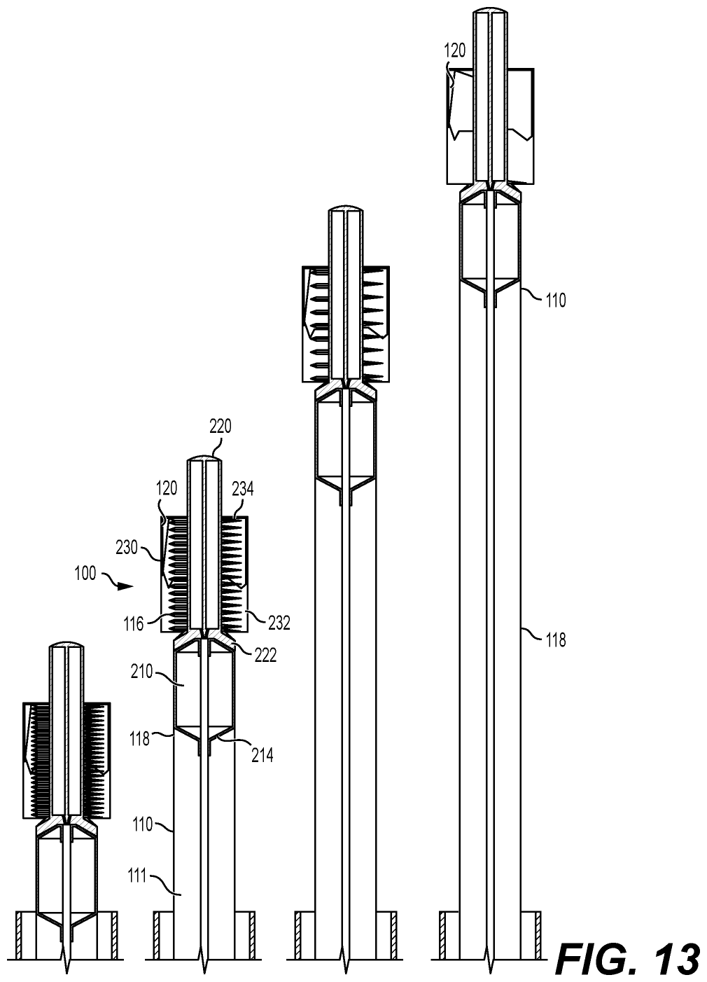

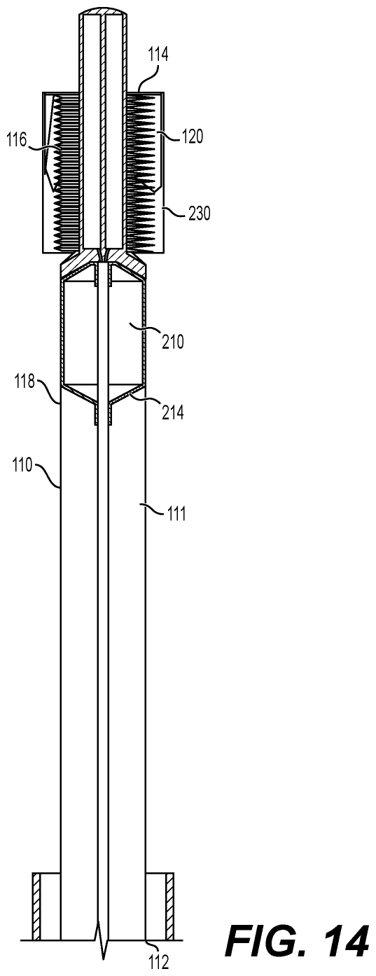

FIGS. 13 and 14 depict the propulsion of the device into the colon due to inflation of the flexible overtube cavity 111 that causes the folded portion 116 of the flexible overtube 110 to be progressively unfolded from the proximal end to the distal end; the transition between the folded portion 116 to the expanded portion 118 being adjacent the base 222 of the steering mechanism 220 and the distal end 212 of the propulsion balloon. To enable such propulsion, the propulsion balloon 210 may be inflated to establish a seal between the outer wall of the propulsion balloon 210 and the inner wall of the flexible overtube 110, to create a flexible overtube cavity 111. Increased fluid pressure in the flexible overtube cavity Ill will exert a force on the proximal end 214 of the propulsion balloon 210, that will cause the propulsion element 210 and the steering mechanism 220 to move distally within the insufflated colon of the patient. The fluid pressure within the sealed flexible overtube cavity Ill and the delivery assembly cavity 211 of the connected delivery assembly 200 results from pressurized fluid (for example, air, carbon dioxide, water, etc.), delivered through the propulsion pressure port 270 which is connected to a propulsion pressure (P_{Propulsion}) regulator of a fluid control system 300, as discussed in more detail below. The propulsion pressure port 270 may be in the wall of the delivery assembly 200 or the wall of the introducer 150 or may be delivered through a lumen in the multi-lumen catheter. All that is required of the propulsion pressure port 270 is that it connects the fluid control system 300 to either flexible overtube cavity Ill or delivery assembly cavity 211.

As the flexible overtube cavity Ill is inflated, the propulsion pressure (P_{Propulsion}) causes the propulsion balloon 210, along with the attached steering mechanism 220, to advance forward within the flexible overtube 110. Since the proximal end 112 of the flexible overtube 110 is attached to the introducer 150, this advancement of the propulsion balloon 210 and steering mechanism 220 causes the folded portion 116 of the flexible overtube 110 to unfold progressively from the proximal end to the distal end over the base 222 of the steering mechanism 220 and the propulsion balloon 210 to both seal with and pass through the unfolded, i.e., inflated portion, 118 of the flexible overtube 110.

Said differently, the progressive unfolding of the folded portion 116 of the flexible overtube 110 causes the flexible overtube cavity 111 and the inflated portion 118 of the flexible overtube 110 to extend in length, thus causing the distal end 114 of the flexible overtube 110, as well as the propulsion balloon 210 and steering mechanism 220, to advance distally into the colon. This deployment of the flexible overtube 110 can be referred to as progressive actuation or controlled deployment of the flexible overtube 110. To be clear, the entire length of the flexible overtube 110 is not inflating/unfolding at the same time. Instead, the flexible overtube 110 is inflating/unfolding progressively from the proximal end 112 of the flexible overtube 110 to the distal end 114 of the flexible overtube 110. That is, the folded portion 116 of the flexible overtube 110 contained within the containment member 230 is advanced forward and unfolded portion 118 of the flexible overtube 110 becomes stationary with respect to the colon upon unfolding. This can also be visualized as the flexible overtube 110 expanding via tip-growth, and as a result the device is not susceptible to looping. This progressive unfolding from proximal end to distal end is a key functional feature of the deployment device 1000.

As the unfolding progresses, the fold containment member 230 gets depleted of the folded portion 116 of the flexible overtube 110. When the folded portion 116 becomes completely unfolded, travel of the flexible overtube 110 ends, and only the fixation balloon 120 remains distal to the base 222 of the steering mechanism 220. In one embodiment, the flat sheet 1201 that is welded to or otherwise attached to flat sheet 1202, to create the deflated fixation balloon 120, may have an inner diameter that is smaller than an outside diameter of the base 222. Such an arrangement prevents the base 222 of the steering mechanism 220 or the propulsion balloon 210 from travelling past the fixation balloon 120. As noted previously, inflation of the fixation balloon 120 results in the frictional fixation of the flexible overtube 110 within the patient's colon. Removal of the delivery assembly 200 from the overtube cavity 111 occurs prior to insertion of a colonoscope.

The bend direction of the steering mechanism 220 can be controlled by independently controlling the pressure applied to the three chambers 224, 225 and 226 by means of an electro-pneumatic or electro-hydraulic pressure control system 300. The internal pressure of the three steering chambers 224, 225 and 226 can be controlled independently through the steering pressure lumens 244, 245 and 246 of the multi-lumen catheter 240 which are connected to steering control valves 326, 327 and 328 in the fluid control system 300.

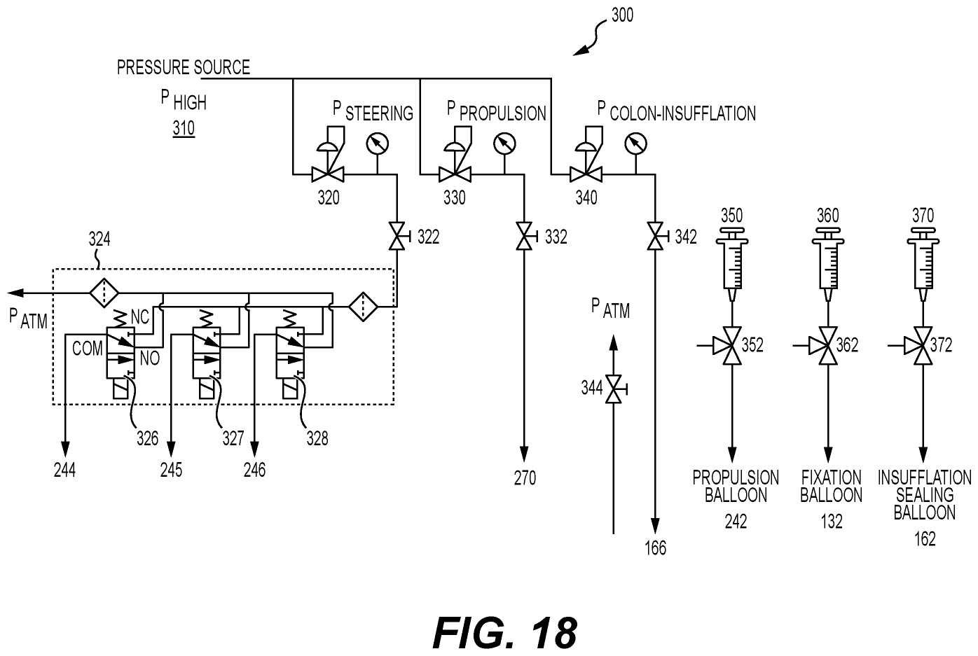

Referring to FIG. 18, the fluid control system 300 includes a source or supply of pressurized fluid 310. The fluid may be air, carbon dioxide, water or any suitable fluid. The source of pressurized fluid 310 is in fluid communication with a steering pressure regulator 320, a propulsion pressure regulator 330, and a colon insufflation pressure regulator 340, which regulate the steering pressures, propulsion pressure, and colon insufflation pressure, respectively. Flow of pressurized fluid from the steering pressure regulator 320 is routed to a steering control valve manifold 324 through a first on/off valve 322. The steering control valve manifold 324 includes three electronically controlled ON/OFF solenoid valves 326, 327, 328. In some embodiments, these valves may be configured for ON/OFF switching control as described below.

FIG. 10 illustrates an exemplary steering control valve schedule which controls the order in which pressure is applied to the three steering chambers 224, 225 and 226. The valve schedule represented in FIG. 10 shows that the steering control valves are cyclically switched on and off in the order 326, followed by 327, and then followed by 328. This will result in rotation of the steering mechanism 220 in one direction (e.g. clockwise rotation). Reversing the order in which the valves are actuated (that is 328, followed by 327, and then followed by 326) will reverse the direction of rotation of the steering actuator (e.g. counterclockwise rotation). Forces exerted by the colon wall on the steering actuator are dependent on direction of rotation. Switching the direction of rotation changes the direction of the forces exerted on the steering actuator. This permits steering mechanism 220 to steer the delivery assembly 200 past obstacles and find a path of least resistance as it advances into the tortuous colon. This prevents the propulsion balloon 210 and steering actuator 220 from becoming stuck or remaining stuck during insertion, and enables automated deployment of the device into the colon

The amplitude of the steering actuator displacement can be controlled by the steering pressure (P_{Steering}) that is controlled by the steering pressure regulator 320. The frequency of the rotation can be controlled by changing the period (T) of the valve schedule as indicated in exemplary valve schedule in FIG. 10. Direction of rotation can be changed by switching the order of valves in the valve schedule, as mentioned above. As indicated on the exemplary valve schedule in FIG. 10, two valves may be ON simultaneously for a certain duration (The overlap fraction (T_{Overlap}/T) can be changed to control the continuity (jerkiness or smoothness) of the steering actuator rotation.

In another embodiment, the valves may be controlled using pulse width modulation (PWM) to control the pressure delivered to the three separate internal chambers 224, 225 and 226 of the steering mechanism 220. A physician may steer based on location, orientation and trajectory feedback from the electromagnetic (EM) sensor or another feedback mechanism.

Propulsion pressure regulator 330 regulates the propulsion pressure (P_{Propulsion}) delivered to the delivery assembly cavity 211 and flexible overtube cavity Ill proximal to the propulsion balloon 210 to control propulsion of the propulsion balloon 210 and, ultimately, unfolding of the flexible overtube 110. The flow is routed through a second on/off valve 332.

Colon insufflation pressure regulator 340 regulates the colon insufflation pressure (P_{colon-insufflation}) The flow is routed to the colon through a third on/off valve 342. The system may also include a pressure relief valve 344 to ensure the pressure in the colon does not exceed the safe pressure limits. The fluid control system 300 may also include a first syringe 350 configured to inflate and deflate the propulsion balloon 210 through a first three-way stopcock valve 352 and propulsion balloon inflation lumen 242; a second syringe 360 configured to inflate and deflate the fixation balloon 120 through a second three-way stopcock valve 362 and fixation balloon inflation port 132; and a third syringe 370 configured to inflate and deflate the insufflation sealing balloon 160 through a third three-way stopcock valve 372 and insufflation sealing balloon port 162.

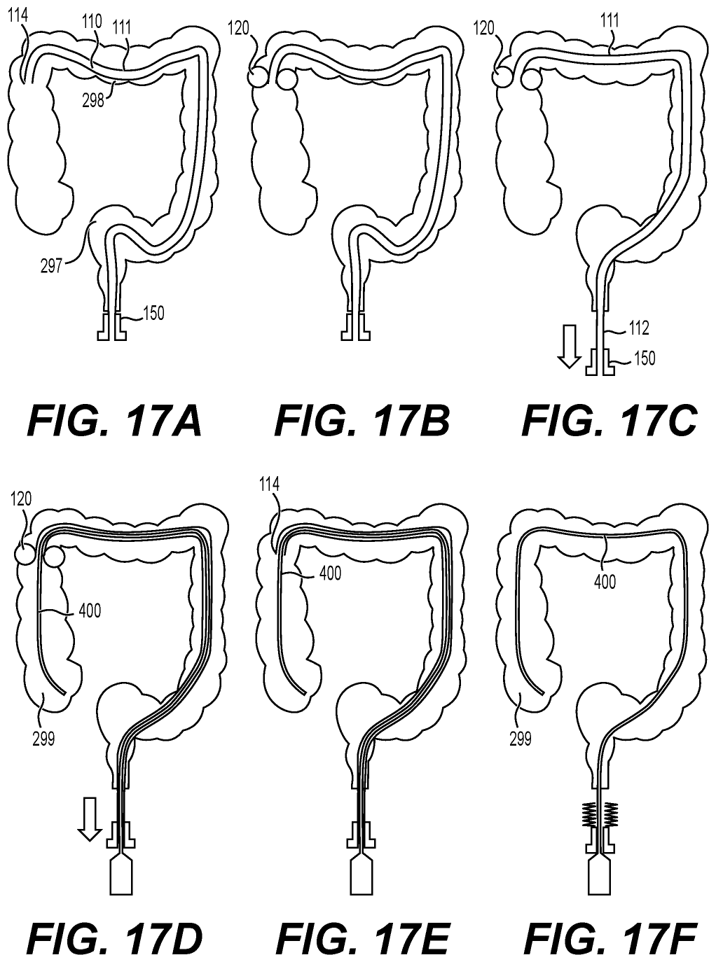

Referring now to FIG. 17, utilization of the flexible overtube 110 to assist with insertion of a colonoscope 400 is described. As shown in FIG. 17A, the introducer 150 is inserted in the anus and the flexible overtube 110 is deployed to a desired position in the colon. While other portions of the device 1000 remain coupled to the overtube 110 at this point, only the overtube 110 is shown for purposes of clarity in FIG. 17. FIG. 17B illustrates inflation of the fixation balloon 120 after deployment of the flexible overtube 110 in the colon. In the illustrated embodiment, the fixation balloon 120 is toroidal/donut-shaped.

As shown in FIG. 17C, once the fixation balloon 120 is inflated to secure the distal end 114 of the flexible overtube 110 at the desired position in the colon, a proximal ‘pulling’ force, illustrated by the arrow, may be exerted on the introducer 150 or any other structure connected to the proximal end 112. Since the distal end of the flexible overtube 110 is held in place relative to the colon by balloon 120, this pulling force introduces tension in the flexible overtube 110. FIGS. 17A and 17B show the sigmoid colon 297 and transverse colon 298 in a “looped” configuration. Applying tension in the overtube 110 will eliminate this “looping”, as seen in FIG. 17C. This is referred to as “shortening the colon” as removal of the “looping” in the sigmoid colon 297 and transverse colon 298 results in the colon being shortened. Typically, removal of the delivery assembly 200 can be accomplished at any point after inflation of balloon 120 but before insertion of scope 400. That said, any structural element in the overtube 110 is removed therefrom prior to insertion of scope 400. The ‘shortening’ of the colon facilitates straightforward insertion of the colonoscope 400. This reduces mucosal stretching and patient pain. This also eliminates musculoskeletal loads on endoscopists and nurses during colonoscope insertion reducing the risk of endoscopy-related injuries.

In one embodiment, where the propulsion element comprises a propulsion balloon 210, the propulsion balloon 210 is deflated and the delivery assembly 200 is removed from the guiding assembly 100 by pulling on the catheter 240, thus leaving only the flexible overtube 110 and fixation balloon 120 in the colon. As noted previously, the fold containment member 230 may invert inside-out to allow for easy removal of the delivery assembly 200 from the guiding assembly 100. With the delivery assembly 200 removed, the physician or nurse can insert a colonoscope 400 through the flexible overtube 110 to a location in the colon beyond the fixation balloon 120, as seen in FIG. 171).

FIG. 17D shows a distal end of colonoscope 400 extending beyond the distal end of the flexible overtube 110, i.e., distally of fixation balloon 120, to or close to the caecum 299. Once the colonoscope 400 has reached the caecum 299, the fixation balloon 120 is deflated, permitting withdrawal of overtube 110. Overtube 110 may be withdrawn at any point after scope placement up to scope removal. In any case, inspection of the colonic mucosa is performed during scope withdrawal, which is standard of care and, the flexible overtube 110 will not interfere with colonic mucosal inspection. In one alternative embodiment, the fixation balloon 120 may be reinflated to allow for repeated insertions of colonoscope 400 and/or insertion(s) of alternative tools, instruments, or endoscopes, that may be required during interventional procedures. The reinflation may be at a site proximal to the intervention site, achieved by pulling a portion of the overtube 110 out of the colon while fixation balloon 120 is deflated. FIG. 17E shows balloon 120 deflated with overtube 110 still in place. FIG. 17F shows colonoscope 400 remaining in place while overtube 110 has been completely withdrawn from the colon.

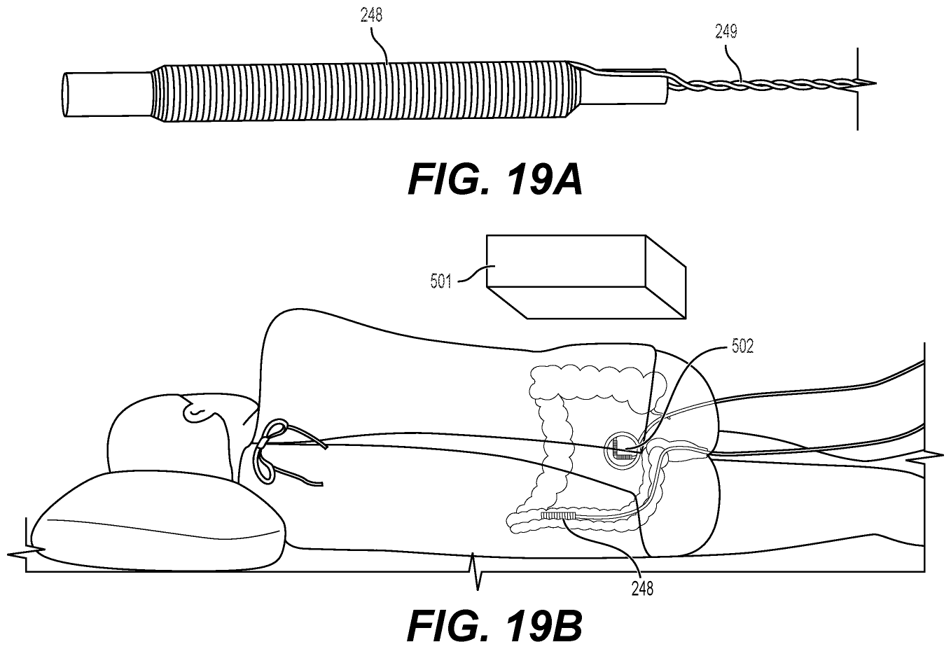

The electromagnetic (EM) tracking system consists of an EM Field generator 501, a 5-DOF (degree of freedom) device mounted EM sensor 248 and a 6-DOF (degree of freedom) patient mounted EM sensor 502, as shown in FIG. 19B. In FIG. 19B, most of the apparatus encompassing the EM coil 248 is removed for clarity. The field generator 501 can take the form of a wide plate placed below the patient or by a planar field generator which may be positioned above the patient using a positioning arm, as shown in FIG. 19B. FIG. 19A is a closeup view of an embodiment comprising a device mounted electromagnetic (EM) coil 248 that may be embedded in any distal location of the guiding assembly 100 or a distal location of the delivery assembly 200 to travel along with the distal end thereof. In FIGS. 8A and 11, the EM coil 248 is shown embedded inside the propulsion balloon 210, embedded within the propulsion balloon inflation lumen 242. The axis of the EM coil 248 is oriented along the axis of the propulsion balloon 210. In an alternative embodiment, the EM coil 248 may be embedded within a lumen of the multi-lumen catheter 240 or wrapped around the multi-lumen catheter 240 or embedded adjacent to the multi-lumen catheter 240. In an embodiment, the EM coil 248 may include a wire wrapped around a solid or hollow ferromagnetic core for optimal sensitivity. The EM coil 248 may be connected to a twisted wire cable 249 which may be connected to a system control unit of an EM tracking system. The twisted wire cable 249 can be routed through the propulsion balloon inflation lumen 242 in a 4-lumen catheter or can be routed through a separate lumen in a 5-lumen catheter. In an alternate embodiment, the twisted wire cable may be routed alongside the multi-lumen catheter. In an alternate embodiment, the EM coil 248 or an additional EM coil may be incorporated within the steering device actuator, preferably at the distal end of the actuator. A single EM coil will provide five degrees-of-freedom feedback on the location and orientation of the distal end of the device.

In conjunction with an EM tracking system, this device mounted EM coil 248 provides 5-DOF (degree of freedom) feedback on the location (x, y, z) and orientation (pitch and yaw) of the propulsion balloon 210 relative to the EM field generator 501. A patient mounted 6-DOF EM sensor 502 containing two EM coils is provided, and attached to the patient during the procedure, for providing 6-DOF location and orientation relative information to the EM field generator 501. The EM tracking system calculates the propulsion balloon location and orientation relative to the patient reference sensor 502. This calculation accounts for any movement of the patient of the field generator 501 during the procedure.

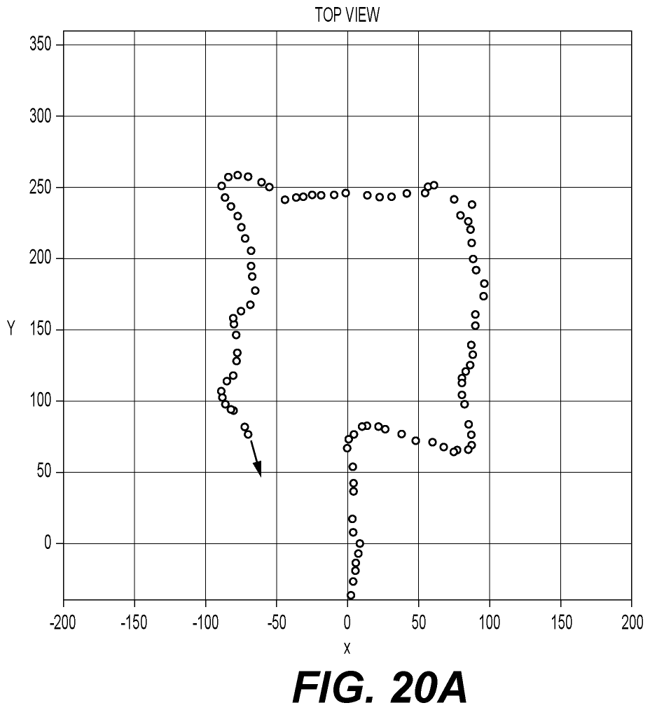

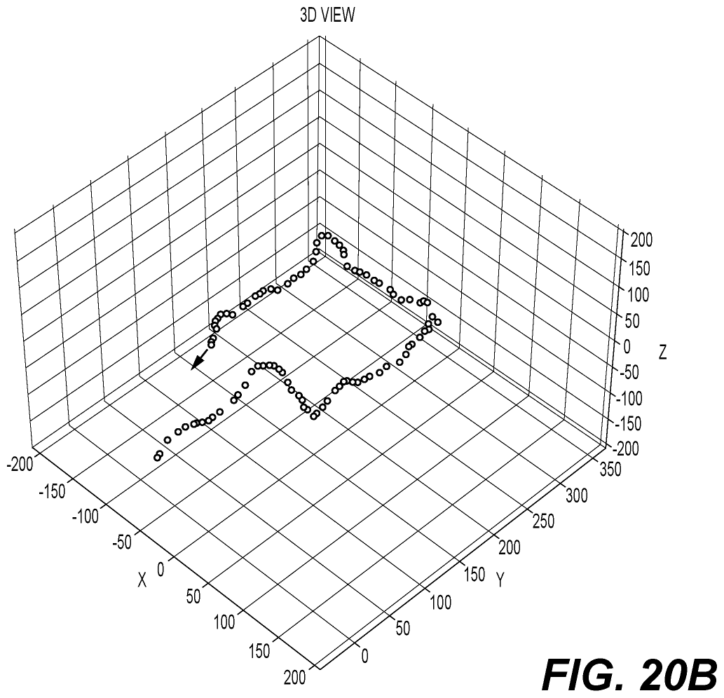

The EM tracking system also generates images to display the current location, current orientation and the trajectory followed by the device mounted EM sensor 248 relative to the patient mounted EM sensor 502. FIGS. 20A and 20B are exemplary 2D and 3D, respectively, graphical outputs of the EM tracking system that track the movement of the device mounted EM coil 248 inside the colon of the patient relative to the patient mounted sensor 502. These outputs permit real time tracking of the EM coil and, thus, any apparatus associated therewith.

In summary, this disclosure describes efficient packaging of a flexible overtube 110 by means of origami fold patterns (or patterns derived from stable inextensional post-buckling patterns of thin-walled cylinders under axial compression or combined axial-torsional loading), carrying the efficiently packaged overtube 110 at the distal end within a fold containment mechanism (as opposed to the tube being stored in the base for everting tube mechanisms), and progressive actuation/progressive unfolding of the flexible overtube 110 from the proximal end 112 to the distal end 114. This packaging offers a convenient way to deliver the propulsion and steering mechanism to deploy the flexible overtube 110.

While multiple non-limiting embodiments have been presented in the foregoing detailed description, it should be appreciated that a vast number of variations exist. It should also be appreciated that the exemplary embodiment or exemplary embodiments are only examples, and are not intended to limit the scope, applicability, or configuration of the disclosure in any way. Rather, the foregoing detailed description will provide those skilled in the art with a convenient road map for implementing the exemplary embodiment or exemplary embodiments. It should be understood that various changes can be made in the function and arrangement of elements without departing from the scope of the disclosure as set forth in the appended claims and the legal equivalents thereof.

What is claimed is:

1. A deployment device, comprising:
a flexible overtube having a folded portion and an unfolded portion, a proximal end of the flexible overtube comprising the unfolded portion and a distal end of the overtube comprising the folded portion;
an introducer fixedly coupled to the proximal end of the flexible overtube;
a propulsion element coupled to a distal end of the unfolded portion of the flexible overtube, wherein the unfolded portion of the overtube and the propulsion element forms an enclosed tubular cavity; and
wherein, the propulsion element is configured to progressively unfold the folded portion of the flexible overtube from a folded portion proximal end to a folded portion distal end, to extend the length of the unfolded portion of the flexible overtube and the tubular cavity, further wherein the propulsion element advances distally relative to the introducer, responsive to a force exerted on the propulsion element.

2. The deployment device of claim 1, wherein a fold pattern of the folded portion of the flexible overtube comprises stable inextensional post-buckling patterns of thin-walled cylindrical tubes under axial compression or combined axial-torsional loading.

3. The deployment device of claim 1, wherein the propulsion element includes a propulsion balloon that may be inflated to form a seal at an inner surface of the unfolded portion of the flexible overtube adjacent a distal end of the unfolded portion.

4. The deployment device of claim 1, further comprising a containment element configured to axially contain the folded portion of the flexible overtube.

5. The deployment device of claim 1, further comprising a steering element coupled to the propulsion element and configured to interact with an environment to change a direction of advancement of the flexible overtube.

6. The deployment device of claim 5 wherein the steering element comprises a flexible element including two or more internal chambers, with a base of the flexible element coupled to the propulsion element and the flexible element extending through the folded portion of the flexible overtube, wherein each internal chamber may be selectively pressurized and depressurized, further wherein selective pressurization of the chambers results in the flexible element bending in a steering direction, the deployment device further comprising a multi-lumen catheter supplying a pressurized fluid to each internal chamber, the catheter extending to the introducer and a fluid supply system adapted to supply fluid pressure to the chambers.

7. The deployment device of claim 6, wherein the flexible element is a cylinder that includes three or more internal chambers symmetrically arranged circumferentially and reinforced by circumferentially oriented fibers, further wherein sequential pressurization and depressurization of the chambers results in a circular motion of the steering actuator.

8. The deployment device of claim 6, wherein the multi-lumen catheter is stored inside a catheter holder having a cavity adapted to contain a pressurized fluid.

9. The deployment device of claim 1, wherein the deployment device being configured for deployment in a patient's colon, the deployment device further comprising:
a fixation element attached to the distal end of the flexible overtube and being configured to maintain the distal end of the flexible overtube at a deployment location.

10. The deployment device of claim 9, wherein the propulsion element is configured to be removable from the flexible overtube, creating a tubular cavity within the patient's colon from the introducer to the deployment location and being configured to guide an endoscope to the deployment location.

11. The deployment device of claim 1, wherein the deployment device being configured for deployment in a patient's colon, the deployment device further comprising:
a toroidally shaped fixation balloon attached to the distal end of the flexible overtube and the fixation balloon being configured to be folded around the folded portion of the flexible overtube, the fixation balloon also being configured to be inflated so as to frictionally engage an inner surface of the patient's colon and maintain the distal end of the flexible overtube at a deployment location; and
an inflation channel attached to the fixation balloon and configured to inflate and deflate the fixation balloon.

12. The deployment device of claim 1, wherein the deployment device being configured for deployment in a patient's colon, the deployment device further comprising:
an insufflation sealing member attached to the introducer, the insufflation sealing member configured to create a seal between the introducer and the patient's colon;
a colon insufflation port adjacent to and located distally of the insufflation sealing member, wherein the insufflation port is configured to supply a pressurized fluid to the patient's colon to insufflate the patient's colon.

13. The deployment device of claim 1, wherein the deployment device being configured for deployment in a patient's colon, the deployment device further comprising:
a steering element coupled to the propulsion element and configured to interact with an inner surface of the patient's colon to periodically change a direction of advancement of the flexible overtube.

14. The deployment device of claim 13, wherein the steering element is configured to periodically rotate clockwise or counterclockwise to periodically change a direction of advancement of the flexible overtube.

15. A method for guiding an endoscope to an in vivo deployment location in a patient's colon, the method comprising:
inserting an introducer into a proximal end of the patient's colon;
fixing the introducer in place adjacent the proximal end of the colon;
advancing a propulsion assembly distally into the patient's colon, wherein a distal end of a flexible overtube is coupled to the propulsion assembly and a proximal end of the flexible overtube is coupled to the introducer;
deploying the flexible overtube, the flexible overtube having a folded portion and an unfolded portion and deploying the overtube includes extending the unfolded portion by unfolding the folded portion until the distal end of the flexible overtube reaches the deployment location, wherein deploying the overtube results from advancing the propulsion assembly and further wherein deploying the flexible overtube results in an enclosed tubular cavity encompassed by the flexible overtube;
supplying pressurized fluid to the enclosed tubular cavity of the overtube, the pressurized fluid exerting an axial force on a propulsion element of the propulsion assembly and advancing the propulsion assembly distally relative to the introducer; and
inserting the endoscope into the proximal end of the flexible overtube, through the enclosed tubular cavity to the distal end of the overtube and, thus, to the deployment location.

16. The method of claim 15, further comprising:
inflating a fixation balloon attached to the distal end of the flexible overtube, whereby the distal end of the flexible overtube frictionally engages an inner surface of the colon and the flexible overtube is fixed in place in relation to the patient's colon;
deflating the fixation balloon such that the overtube is no longer fixed in place in relation to the patient's colon;
adjusting the position of the flexible overtube relative to the patient's colon; and
reinflating the fixation balloon.

17. The method of claim 15, wherein the propulsion assembly includes a propulsion balloon and the method further comprising:
inflating the propulsion balloon to create a seal between the inflated propulsion balloon and an inner surface of the flexible overtube, wherein the axial force is exerted on a proximal portion of the inflated propulsion balloon.

18. The method of claim 15, wherein the propulsion assembly further comprises a steering element having a proximal end, a distal end and a flexible cylinder enclosing two or more internal chambers, wherein the distal end of the steering element comprises a distal end of the delivery assembly when the delivery assembly is in use, the method further comprising:
steering the propulsion assembly by selectively pressurizing and depressurizing the internal chambers of the steering element, resulting in the flexible cylinder bending in a steering direction, wherein steering includes periodic changes in rotation of the distal end of the steering element resulting in periodic changes in a direction of advancement of the propulsion assembly.

19. The method of claim 15, further comprising:
creating a seal between the introducer and the patient's colon by inflating a sealing member attached to the introducer; and
insufflating the patient's colon by pumping pressurized fluid into the patient's colon distally of the insufflation sealing member through an insufflation port.

20. A method for guiding an endoscope to an in vivo deployment location in a patient's colon, the method comprising:
inserting an introducer and a propulsion assembly into a proximal end of the patient's colon, the propulsion assembly comprising a propulsion element and a flexible overtube;
advancing the propulsion assembly distally into the patient's colon, a distal end of the overtube is connected to the propulsion assembly and a proximal end of the overtube is connected to the introducer such that advancement of the propulsion assembly results in the flexible overtube being deployed and results in an enclosed tubular cavity encompassed by the flexible overtube, wherein the propulsion assembly is advanced to a deployment location in the patient's colon;
inflating a fixation balloon attached to the distal end of the flexible overtube, the fixation balloon configured to maintain the distal end of the overtube at the deployment location;
shortening the patient's colon by exerting a pulling force on the one or both of the flexible overtube and the propulsion element;
removing the propulsion element from the flexible overtube; and
inserting the endoscope into the proximal end of the flexible overtube, through the enclosed tubular cavity to the distal end of the overtube and, thus, to the deployment location.
